(19) United States
(12) Patent Application Publication
(10) Pub. No.: US 2024/0238648 A1
(43) Pub. Date: Jul. 18, 2024

(54) ADAPTIVE WEIGHT PRESCRIPTION SYSTEM AND METHOD FOR RESISTANCE TRAINING

(71) Applicant: Nautilus, Inc., Vancouver, WA (US)

(72) Inventors: Marcus L. Marjama, Vancouver, WA (US); John Lindala, Portland, OR (US)

(73) Assignee: Nautilus, Inc., Vancouver, WA (US)

(21) Appl. No.: 18/095,394

(22) Filed: Jan. 10, 2023

(51) Int. Cl. A63B 24/00; A63B 71/06; G16H 20/30; G16H 40/67

(52) U.S. Cl. CPC A63B 24/0075; A63B 71/0622; G16H 20/30; G16H 40/67; A63B 2024/0068; A63B 2024/0081; A63B 2220/05; A63B 2220/17; A63B 2220/806

(57) ABSTRACT

A computer-implemented method includes generating a workout plan for a user having a user profile. The workout plan includes an exercise selected from a set of exercises stored in the user profile. The exercise has a prescribed weight and a prescribed number of exercise repetitions. The method includes capturing user performance data for the exercise during execution of the workout plan by the user in an exercise environment. The user performance data includes an actual weight used in performance of the exercise and an actual number of exercise repetitions by the user. The method includes determining a new value for the prescribed weight based on a stored value of the prescribed weight in the user profile, the prescribed number of exercise repetitions, and the user performance data. The stored value of the prescribed weight in the user profile is adjusted to the new value for the prescribed weight.

### FIELD

The field generally relates to resistance training and to methods and systems for assigning weights in resistance training.

### BACKGROUND

Muscle mass decreases with age, for example, at approximately 3-5% per decade after the age of 30. In the medical community, muscle mass loss due to aging is known as sarcopenia. To preserve or enhance muscle mass, the medical community recommends progressive resistance training. Resistance training (also known as strength training) involves performing exercises that cause muscles to contract against an external resistance, which can be provided by body weight, free weights (such as dumbbells, kettlebells, etc.), weight machines, or resistance bands. The weights are pushed or pulled in order to resist muscle movement. In progressive resistance training, the workout volume (e.g., the weight used in the workout times the number of exercise repetitions in the workout) is gradually increased as strength and endurance improve.

Although there is agreement that resistance training is beneficial, there still remains the obstacles of motivating and guiding participants before, during, and after exercising. Although general information about resistance training is widely available, it is typically static in nature and not personalized to an individual. Thus, there remains room for improvement in resistance training.

### BRIEF DESCRIPTION OF THE DRAWINGS

FIG. 1 is a block diagram of an example system implementing adaptive weight prescription for resistance training.

FIG. 2 is a flow diagram of an example method implementing adaptive weight prescription for resistance training.

FIG. 3 is a flow diagram of an example method for determining a new value for a prescribed weight of an exercise for an optimized user scenario.

FIG. 4 is a flow diagram of an example method for determining a new value for a prescribed weight of an exercise for an overestimated user capability scenario.

FIG. 5 is a flow diagram of an example method for determining a new value for a prescribed weight attribute of an exercise for an alternative user selected lower weight scenario.

FIG. 6 is a flow diagram of an example method for determining a new value for a prescribed weight attribute of an exercise for an alternative user selected higher weight scenario.

FIG. 7 is a flow diagram of an example method implementing initial weight prescription for resistance training.

FIG. 8 is a block diagram of an example computing system in which described technologies can be implemented.

FIG. 9 is a block diagram of an example cloud computing environment that can be used in conjunction with the technologies described herein.

### DETAILED DESCRIPTION

### Example I—Overview

Described herein are technologies providing adaptive weight prescription for resistance training. The technologies provide a system that prescribes weights to use in performing resistance training exercises based on demonstrated user performance. In some examples, the technologies provide an exercise coding system that facilitates swapping of like exercises, which can allow flexibility in configuring workouts.

### Example 2—Example System Implementing Adaptive Weight Prescription for Resistance Training

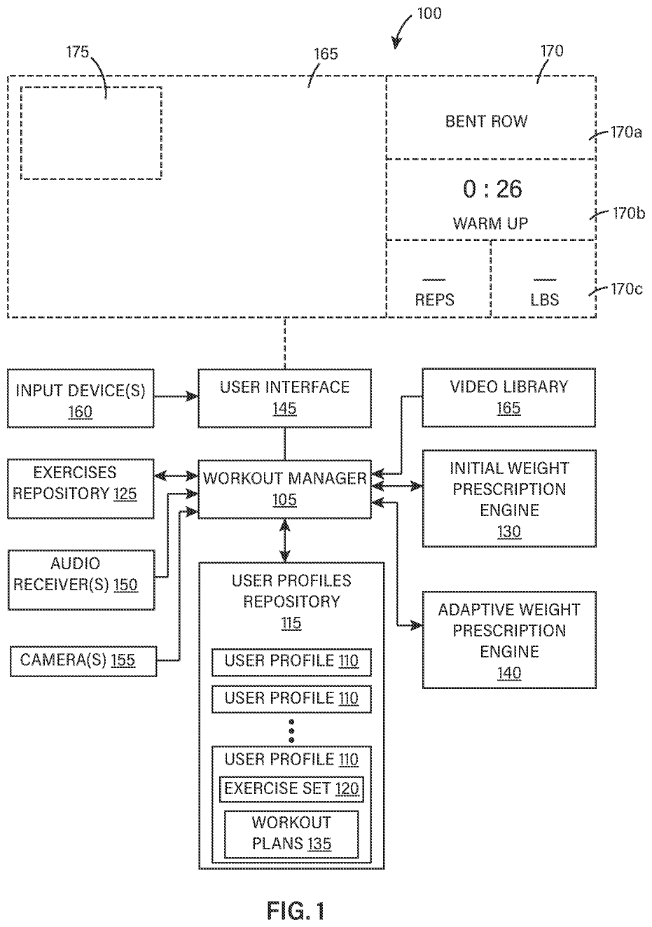

FIG. 1 is a block diagram of an example system 100 implementing adaptive weight prescription for resistance training. In the example, the system 100 can include a workout manager 105 that can generate and manage user profiles 110. The workout manager 105 can store the user profiles 110 in a user profiles repository 115. A user profile 110 can include a set of exercises 120. For example, the workout manager 105 can add the set of exercises 120 to the user profile 110 at the time that the user profile 110 is created. For example, the workout manager 105 can copy the set of exercises 120 from an exercises repository 125 containing a master list of exercises. The exercise 120 can have various attributes, such as exercise code, prescribed weight, prescribed number of exercise repetitions, and capability index (see the exercise structure in Examples 10 and 11). Values for the attributes can be stored in the user profile 110.

The workout manager 105 can generate and manage workout plans 135 for users. The workout plans 135 for a given user can be stored in the user profile 110 associated with the given user. The workout plan 135 can include selected exercises from the set of exercises 120 in the user profile 110 of the given user. The workout manager 105 can capture user performance data (e.g., exercises performed, weights used in the exercises performed, and the number of repetitions of the exercises performed) during execution of the workout plan 135 by the user in an exercise environment. In some examples, the workout manager 105 can store the user performance data in the user profile 110.

The system 100 can include an initial weight prescription engine 130 that determines initial prescribed weights for the set of exercises 120 in a given user profile 110. In some examples, the initial weight prescription engine 130 can accept a user profile 110 from the workout manager 105, determine initial prescribed weights for the set of exercises 120 in the user profile, store the determined initial prescribed weights in the user profile 110 as initial values for the prescribed weights of the set of exercises 120, and return the user profile 110 to the workout manager 105. In some examples, the initial weight prescription engine 130 can include rules to calculate initial prescribed weights for the set of exercises 120 in a user profile 110 based on performance of the user on a set of base movement patterns (see Example 8).

The system 100 can include an adaptive weight prescription engine 140 that determines new values for prescribed weights of exercises performed by the user during workouts based on user performance data captured during the workouts. The adaptive weight prescription engine 140 can accept a user profile 110 from the workout manager 105 and extract a recently completed workout plan 135 from the user profile 110. The adaptive weight prescription engine 140 can extract user performance data for a given exercise performed in the recently completed workout plan 135. The adaptive weight prescription engine 140 can determine a new value for the prescribed weight of the given exercise based on the user performance data. The adaptive weight prescription engine 140 can compute other data for the given exercise, such as the capability index for the given exercise (see Example 11).

The user profile 110 can store data related to the set of exercises 120 (such as values for the attributes of the exercises 120). The user profile 110 can store workout plans 135 generated for the user. The user profile 110 can store a body weight of the user. For example, when a new user profile 110 is created, the workout manager 105 can collect the body weight of the user and store the body weight in the user profile. If the user completes an initial fitness assessment (see Example 8), the user profile 110 can include the user performance data (e.g., exercises performed, weights used in the exercises, and number of repetitions of the exercises completed) captured during the initial fitness assessment. The user profile 110 can include user performance data captured during subsequent workouts.

In some examples, the workout manager 105 can include a user interface 145 (e.g., a graphical user interface) that can be presented on a display for user interaction. In some examples, the workout manager 105 can present a list of user profiles 110 (e.g., active user profiles from the user profile repository 115) on the user interface 145. A user (or a trainer for the user) can select a desired user profile 110 from the list or create a new user profile 110. In some examples, the workout manager 105 can present workout plans 135 associated with a selected user profile 110 on the user interface 145. The user (or the trainer for the user) can select a desired workout plan 135 from the list or create a new workout plan 135. In some examples, the user can edit, delete, or archive the selected workout plan 135 through interaction with the user interface 145.

The system 100 can include one or more devices (e.g., audio receiver(s) 150, camera(s) 155, etc.) to capture commands and data from an exercise environment and/or one or more input devices 160 (e.g., keyboard, touchpad, etc.) to enter data or select controls on the user interface 145. In some examples, the workout manager 105 can receive an output of the audio receiver(s) 150 and process the output to identify commands and/or data from the exercise environment. In some examples, the workout manager 105 can receive video images captured by the camera(s) 155 and process the video images to identify data from the exercise environment. For example, the camera(s) 155 can capture video images of a user during a workout in the exercise environment, and the workout manager 105 can determine the number of repetitions of an exercise performed by the user during the workout from the video images.

In some examples, the system 100 can include a video library 165 having video clips corresponding to the exercises in the exercises repository 125. A video clip corresponding to an exercise can, for example, feature a trainer that guides a user through performance of the exercise. In some examples, when the workout manager 105 generates a workout plan 135, the workout manager 105 can also retrieve the video clips corresponding to the exercises in the workout plan 135 from the video library 165. The workout manager 105 can assemble the video clips into a video package that can be played during execution of the workout plan 135 in an exercise environment.

In some examples, the user interface 145 can have a main content area 165 and a side bar 170. During execution of a workout plan 135, video clips associated with the exercises in the workout plan 135 can be played in the main content area 165. In some examples, a window 175 can be displayed over a portion of the main content area 165. The window 175 can display the user performing the exercises in the exercise environment. For example, video images captured by the camera(s) 155 can be streamed in the window 175. In some examples, the user can start, stop, or pause playing of the videos in the main content area 165 by voice commands or by manually selecting controls on the user interface 145.

In some examples, information about execution of the workout plan 135 can be displayed in the side bar 170. For illustration purposes, the name of the exercise currently performed can be displayed in a top portion 170a of the side bar 170. Information about the progress of the exercise (such as time elapsed since start of the exercise and the current phase of the exercise) can be displayed in a middle portion 170b of the side bar 170. Information about performance of the exercise (such as the number of repetitions completed and the weight used in the exercise) can be displayed in a lower portion 170c of the side bar 170. The information displayed in the side bar 170 can be updated in real time.

The system 100 can be implemented in a computer system or a network of computer systems in the form any of a variety of hardware (e.g., integrated hardware module, standalone device, console, set-top box, or the like). The software components of the system, the user profiles, the workout plans, video clips, and the like can be stored in one or more computer-readable storage media or computer-readable storage devices, and the software components can be executed by one or more processor units. The technologies described herein can be generic to the specifics of operating systems or hardware and can be applied in any variety of environments to take advantage of the described features.

### Example 3—Example Method Implementing Adaptive Weight Prescription for Resistance Training

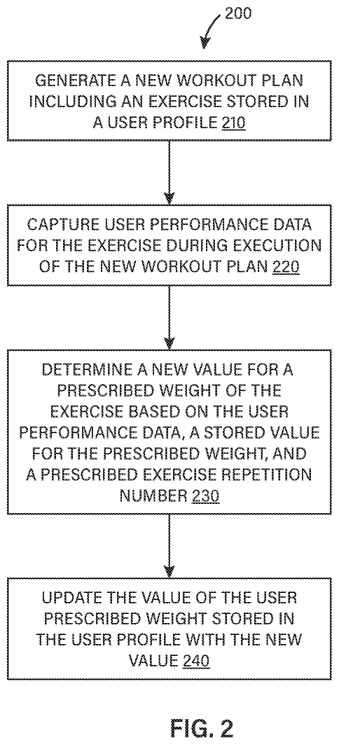

FIG. 2 is a flow diagram of an example method 200 of adaptive weight prescription for resistance training and can be performed, for example, by the system of FIG. 1. The method 200 prescribes weights to use in performing exercises by a user based on previous performance of the user on the exercises. The method 200 assumes that a user profile has been created for the user and that the user profile includes a set of exercises that can be performed by the user in workouts. Operations are illustrated once and each in a particular order in FIG. 2, but the operations may be reordered and/or repeated as desired and appropriate (for example, different operations illustrated as performed sequentially may be performed in parallel as suitable).

At 210, the method can include generating a new workout plan for a user having a user profile. The new workout plan can include one or more exercises selected from a set of exercises stored in the user profile. An exercise can have various attributes, such as an exercise code, a prescribed weight, a prescribed number of exercise repetitions, an exercise multiplier, user capability index, equipment type, etc. (see Examples 10 and 11). A set of weight graduation levels can be associated with the equipment type (see Example 11). Values for the attributes of the exercise can be stored in the user profile in association with the exercise.

In some examples, generating the new workout plan can include creating a blank workout plan and adding one or more exercises to the workout plan from the set of exercises associated with the user profile. In other examples, generating the new workout plan can include presenting previously generated workout plans to a user (e.g., in a user interface on a display), receiving a selected previously generated workout plan from the user, and generating the new workout plan based on the selected previously generated workout plan (e.g., by including the exercises in the selected previously generated workout plan in the new workout plan). In some examples, the user can modify a previously generated workout plan prior to submitting the previously generated workout plan for use in generating the new workout plan. Modifications to a previously generated workout plan can include any of adding one or more exercises to the workout plan, deleting one or more exercises from the workout plan, and swapping an exercise in the workout plan for an equivalent exercise from the set of exercises (see Example 10 for exercise coding that facilitates swapping of exercises).

At 220, the method can include capturing user performance data for exercises during execution of the workout plan in an exercise environment by the user. For a given exercise, the user performance data can include the actual weight the user used in performing the exercise, if the exercise involves weight, and the number of exercise repetitions performed by the user. The user performance data can be captured via various methods, such as by voice commands, by receiving input via a user interface, and/or by capturing video images of the user. For example, at the beginning of an exercise E, the user can announce the weight that the user plans to use in the exercise E, or the user can enter the weight in a user interface using an input device. In one example, after the user completes each set of the exercise E (where a set can have one or more repetitions of the exercise), the user can announce how many exercise repetitions the user completed in the set. In another example, video images of the user can be captured by a hardware camera while the user is performing the exercise E, and the number of repetitions the user completes for the exercise E can be extracted from the video images. For example, the user can wear a visible marker, and displacements of the visible marker in the video images can be used to determine the number of repetitions the user completes for the exercise.

At 230, the method can include determining a new value for the prescribed weight for a given exercise in the workout plan. The new value can be determined based on the stored value of the prescribed weight for the given exercise, the prescribed number of exercise repetitions, and the captured user performance data for the given exercise. The method can also include determining a new value for the user capability index for the exercise. Examples 4-7 describe methods of determining new values for prescribed weight and user capability index for various actual weight and actual number of exercise repetitions scenarios. Operation 230 can be repeated for each exercise in the workout plan.

At 240, the method can include updating the stored value of the prescribed weight to the new value determined in operation 230. The method can also include updating the stored value of the capability index to the new value determined in operation 230. Operation 240 can be repeated for each exercise in the workout plan. The new values for the prescribed weights of the exercises will be available as prescribed starting points for the next performance of the exercises in a workout.

### Example 4—Example Method Implementing Adaptive Weight Prescription for an Optimized User

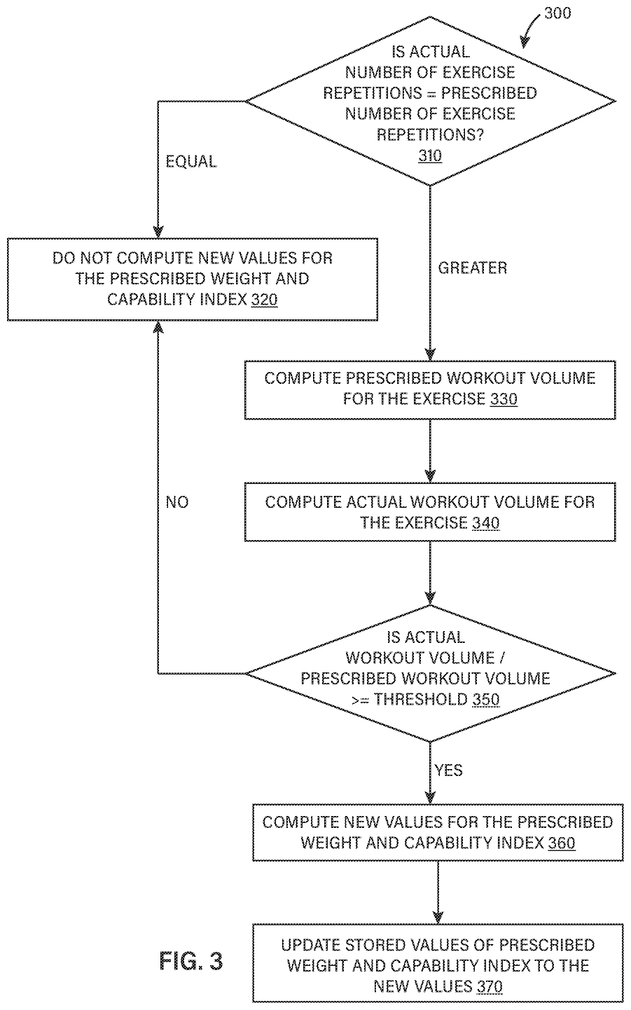

FIG. 3 is a flow diagram of an example method 300 for determining new values for prescribed weight of an exercise after the user performs the exercise in a workout. The method 300 can be performed in operation 230 of the method 200 in FIG. 2. The method 300 can be used for a scenario where the user is performing optimally. The method 300 assumes that the actual number of exercise repetitions performed by the user is equal to or greater than the prescribed number of exercise repetitions and that the actual weight used in performing the exercise is the same as the prescribed weight.

At 310, the method includes determining whether the actual number of exercise repetitions is equal to the prescribed number of exercise repetitions. If the actual number of exercise repetitions is equal to the prescribed number of exercise repetitions, the method ends at 320. If the actual number of exercise repetitions is greater than the prescribed number of exercise repetitions, the method continues at 330.

At 320, new values for the prescribed weight and capability index are not determined. The stored values of the prescribed weight and capability index are left unchanged.

At 330, the method can include computing a prescribed workout volume for the exercise. A workout volume is a measure of the total weight lifted during performance of an exercise. The prescribed workout volume for an exercise can be defined as the prescribed weight for the exercise multiplied by the prescribed number of exercise repetitions for the exercise. The prescribed weight and prescribed number of exercise repetitions for the exercise can be obtained from the user profile associated with the exercise.

At 340, the method can include computing an actual workout volume for the exercise. The actual workout volume can be defined as the actual weight used in performing the exercise times the actual number of exercise repetitions performed. The actual weight and actual number of exercise repetitions can be obtained from the user performance data captured while the user performed the exercise.

At 350, the method can include determining if a ratio of the actual workout volume to the prescribed workout volume is equal to or greater than a threshold T1. In one example, threshold T1 can be in a range from 1 to 1.5. In a particular example, the threshold T1 can be 1.2. If the ratio of the actual workout volume to the prescribed workout volume is equal to or greater than the threshold T1, the method continues to 360. If the ratio of the actual workout volume to the prescribed workout volume is less than the threshold T1, the method ends at 320.

At 360, the method includes determining new values for the prescribed weight and capability index of the exercise. In one example, a new value for the prescribed weight is calculated as the stored value (e.g., stored in the user profile) of the prescribed weight incremented by one weight graduation level. For example, given a set of weight graduation levels {w₁<w₂< . . . <wₖ₋₁<wₖ<wₖ₊₁< . . . <wₙ₋₁<wₙ} associated with the exercise, if the stored value is weight wₖ in the set of weight graduation levels, then the new value for the prescribed weight can be weight wₖ₊₁. (The stored value can be rounded to the nearest weight in the set of weight graduation levels for the purpose of determining the new value for the prescribed weight.) In one example, the new value for the capability index can be determined as the new value for the prescribed weight divided by the exercise multiplier associated with the exercise.

At 370, the stored value for the prescribed weight is updated to the new value for the prescribed weight. If the new value for the capability index is less than the stored value for the capability index, then the stored value for the capability index is updated to the new value for the capability index.

### Example 5—Example Method Implementing Adaptive Weight Prescription for an Overestimation of Capability

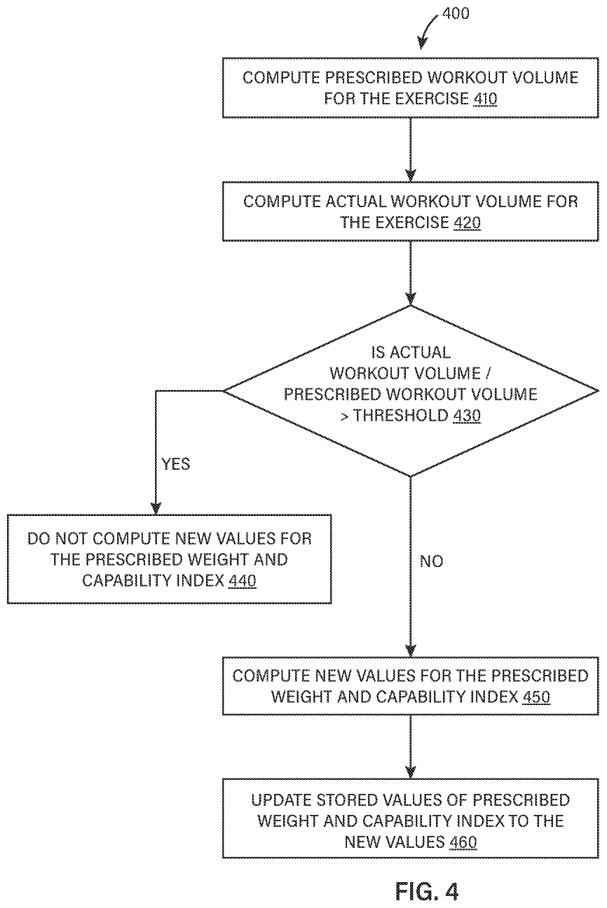

FIG. 4 is a flow diagram of an example method 400 for determining a new value for a prescribed weight of an exercise after the user performs the exercise in workout. The method 400 can be performed in operation 230 of the method 200 of FIG. 2. The method 400 can be used for a scenario where the capability of the user has been overestimated. The method 400 assumes that the actual number of exercise repetitions performed by the user is less than the prescribed number of exercise repetitions and that the actual weight used in performing the exercise is the same as the prescribed weight.

At 410, the method can include computing a prescribed workout volume for the exercise. A workout volume is a measure of the total weight lifted during performance of an exercise. The prescribed workout volume for an exercise can be defined as the prescribed weight for the exercise times the prescribed number of exercise repetitions for the exercise. The prescribed weight and prescribed number of exercise repetitions for the exercise can be obtained from the user profile associated with the exercise.

At 420, the method includes computing an actual workout volume for the exercise. The actual workout volume can be defined as the actual weight used in performing the exercise times the actual number of exercise repetitions performed. The actual weight and actual number of exercise repetitions can be obtained from the user performance data captured while the user performed the exercise.

At 430, the method can include determining if a ratio of the actual workout volume to the prescribed workout volume is greater than a threshold T2. In one example, the threshold T2 can be in a range from 0.6 to 0.8 (e.g., 0.75 or the like). In a particular example, the threshold T2 can be 0.75. If the ratio of the actual workout volume to the prescribed workout volume is greater than the threshold T2, the method ends at 440. Otherwise, if the ratio of the actual workout volume to the prescribed workout volume is equal to or less than the threshold T2 (which means that the capability of the user has been overestimated), the method continues to 450. Such a threshold can be helpful to determine if the user is significantly underperforming the recommended volume, which can be useful for reducing the future prescription and finding a proper recommendation.

At 440, new values for the prescribed weight and capability index are not determined. The stored values of the prescribed weight and capability index are left unchanged.

At 450, the method includes determining new values for the prescribed weight and capability index of the exercise. In one example, a new value for the prescribed weight is calculated as the stored value (e.g., stored in the user profile) of the prescribed weight decremented by one weight graduation level. For example, given a set of weight graduation levels {w₁<w₂< . . . <wₖ₋₁<wₖ< . . . <wₙ₋₁<wₙ} associated with the exercise, if the stored value is weight wₖ in the set of weight graduation levels, then the new value for the prescribed weight can be weight wₖ₋₁. (The stored value can be rounded to the nearest weight in the set of weight graduation levels for the purpose of determining the new value for the prescribed weight.) In one example, the new value for the capability index can be determined as the new value for the prescribed weight divided by the exercise multiplier associated with the exercise.

At 460, the stored value for the prescribed weight is updated to the new value for the prescribed weight. If the new value for the capability index is less than the stored value for the capability index, then the stored value for the capability index is updated to the new value for the capability index.

### Example 6—Example Method Implementing Adaptive Weight Prescription for Lower Weight Selection

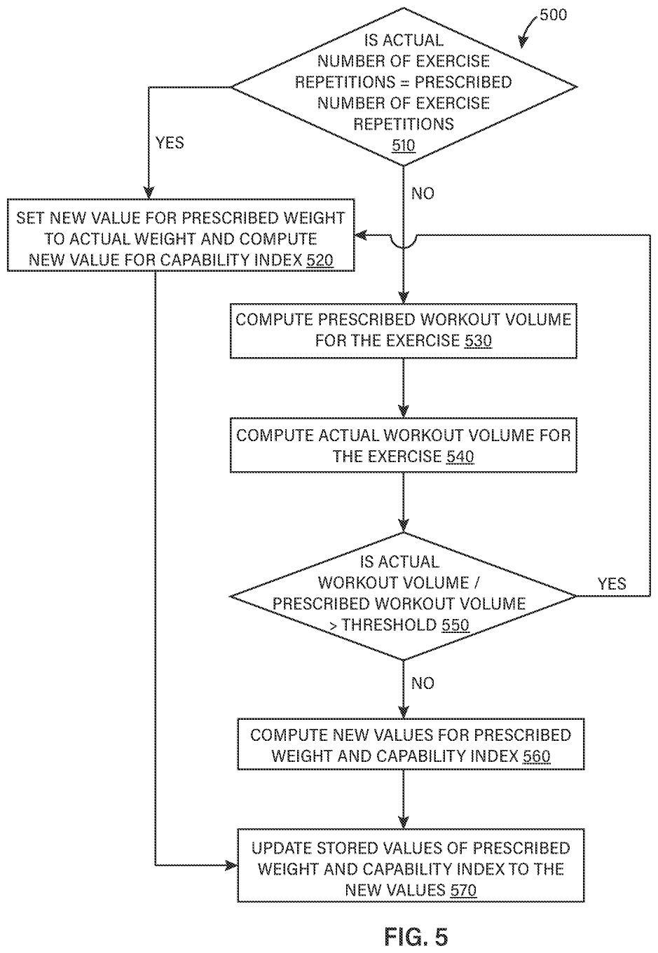

FIG. 5 is a flow diagram of an example method 500 for determining a new value for a prescribed weight of an exercise after the user performs the exercise in a workout. The method 500 can be performed in operation 230 of the method 200 in FIG. 2. The method 500 can be used for a scenario where the user uses an actual weight that is different from the prescribed weight for the exercise. The method 500 assumes that the actual number of exercise repetitions performed by the user is less than the prescribed number of exercise repetitions and that the actual weight used in performing the exercise is lower than the prescribed weight.

At 510, the method includes determining whether the actual number of exercise repetitions is equal to the prescribed number of exercise repetitions. If actual number of exercise repetitions is equal to the prescribed number of exercise repetitions, the method continues to 520. If the actual number of exercise repetitions is not equal to the prescribed number of exercise repetitions, the method continues to 530.

At 520, the method includes determining new values for the prescribed weight and capability index. In one example, the new value for the prescribed weight is set to the actual weight used in performing the exercise by the user. In one example, the new value for the capability index can be determined as the new value for the prescribed weight divided by the exercise multiplier associated with the exercise. After operation 520, the method continues to 570 to store the new values.

At 530, the method can include computing a prescribed workout volume for the exercise. A workout volume is a measure of the total weight lifted during performance of an exercise. The prescribed workout volume for an exercise can be defined as the prescribed weight for the exercise times the prescribed number of exercise repetitions for the exercise. The prescribed weight and prescribed number of exercise repetitions for the exercise can be obtained from the user profile associated with the exercise.

At 540, the method can include computing an actual workout volume for the exercise. The actual workout volume can be defined as the actual weight used in performing the exercise times the actual number of exercise repetitions performed. The actual weight and actual number of exercise repetitions can be obtained from the user performance data captured while the user performed the exercise.

At 550, the method can include determining if a ratio of the actual workout volume to the prescribed workout volume is greater than a threshold T3. In one example, threshold T3 can be in a range from 0.4 to 0.6. In a particular example, threshold T3 can be 0.5. If the ratio of the actual workout volume to the prescribed workout volume is greater than the threshold T3, the method returns to 520. If the ratio of the actual workout volume to the prescribed workout volume is not greater than the threshold, the method continues to 560.

At 560, the method includes determining new values for the prescribed weight and capability index. In one example, a new value for the prescribed weight is calculated as the actual weight decremented by one weight graduation level. For example, given a set of weight graduation levels {w₁<w₂< . . . <wₖ₋₁<wₖ<wₖ₊₁< . . . <wₙ₋₁<wₙ} associated with the exercise, if the actual weight is weight wₖ in the set of weight graduation levels, then the new value for the prescribed weight can be weight wₖ₋₁. (The stored value can be rounded to the nearest weight in the set of weight graduation levels for the purpose of determining the new value for the prescribed weight.) In one example, the new value for the capability index can be determined as the new value for the prescribed weight divided by the exercise multiplier associated with the exercise.

At 570, the stored value for the prescribed weight is updated to the new value for the prescribed weight. If the new value for the capability index is less than the stored value for the capability index, then the stored value for the capability index is updated to the new value for the capability index.

### Example 7—Example Method Implementing Adaptive Weight Prescription for Higher Weight Selection

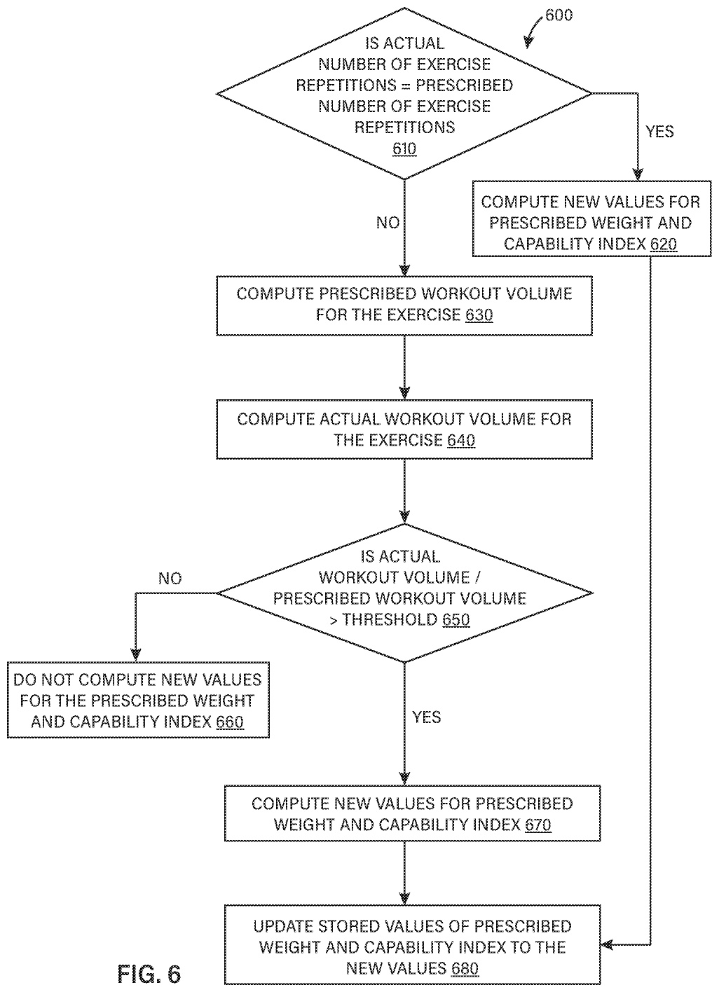

FIG. 6 is a flow diagram of an example method 600 for determining new values for a prescribed weight of an exercise after the user performs the exercise in a workout. The method can be performed in operation 230 of the method 200 in FIG. 2. The method 600 can be used for a scenario where the user uses an actual weight that is different from the prescribed weight for the exercise. The method 600 assumes that the actual number of exercise repetitions performed by the user is less than the prescribed number of exercise repetitions and that the actual weight used in performing the exercise is higher than the prescribed weight.

At 610, the method includes determining whether the actual number of exercise repetitions is equal to the prescribed number of exercise repetitions. If the actual number of exercise repetitions is equal to the prescribed number of exercise repetitions, the method continues to 620. If the actual number of exercise repetitions is not equal to the prescribed number of exercise repetitions, the method continues to 630.

At 620, the method includes determining new values for the prescribed weight and capability index. In one example, a new value for the prescribed weight is calculated as the actual weight incremented by one weight graduation level. For example, given a set of weight graduation levels {w₁<w₂< . . . <wₖ₋₁<wₖ<wₖ₊₁< . . . <wₙ₋₁<wₙ} associated with the exercise, if the actual weight is weight wₖ in the set of weight graduation levels, then the new value for the prescribed weight can be weight wₖ₊₁. (The stored value can be rounded to the nearest weight in the set of weight graduation levels for the purpose of determining the new value for the prescribed weight.) In one example, the new value for the capability index can be determined as the new value for the prescribed weight divided by the exercise multiplier associated with the exercise. The method continues to 680 to store the new values.

At 630, the method can include computing a prescribed workout volume for the exercise. A workout volume is a measure of the total weight lifted during performance of an exercise. The prescribed workout volume for an exercise can be defined as the prescribed weight for the exercise times the prescribed number of exercise repetitions for the exercise. The prescribed weight and prescribed number of exercise repetitions for the exercise can be obtained from the user profile associated with the exercise.

At 640, the method can include computing an actual workout volume for the exercise. The actual workout volume can be defined as the actual weight used in performing the exercise times the actual number of exercise repetitions performed. The actual weight and actual number of exercise repetitions can be obtained from the user performance data captured while the user performed the exercise.

At 650, the method can include determining if a ratio of the actual workout volume to the prescribed workout volume is greater than a threshold T4. In one example, the threshold T4 can be in a range from 1 to 1.2. In a particular example, the threshold T4 can be 1.0. If the ratio of the actual workout volume to the prescribed workout volume is greater than the threshold T4, the method continues at 670. If the ratio of the actual workout volume to the prescribed workout volume is not greater than the threshold T4, the method continues at 660.

At 660, new values for the prescribed weight and capability index are not determined. The stored values of the prescribed weight and capability index are left unchanged.

At 670, the method includes determining new values for the prescribed weight and capability index. In one example, the new value for the prescribed weight is set to the actual weight used in performing the exercise by the user. In one example, the new value for the capability index can be determined as the new value for the prescribed weight divided by the exercise multiplier associated with the exercise.

At 680, the stored value for the prescribed weight is updated to the new value for the prescribed weight. If the new value for the capability index is less than the stored value for the capability index, then the stored value for the capability index is updated to the new value for the capability index.

### Example 8—Example Initial Fitness Assessment

An initial fitness assessment of a user can be based on a set of base movement patterns. In one example, the set of base movement patterns can include push, pull, core, squat, and hinge. The movement patterns can have associated weight formulas used to determine the weight the user uses when performing the base movement pattern in the initial fitness assessment. In some examples, the weight formulas can be based on the body weight of the user.

The set of base movement patterns can be found in various resistance training exercises. Table 1 below gives an example of resistance training exercises corresponding to the set of base movement patterns. Also shown in Table 1 are example weight formulas corresponding to the set of base movement patterns. The weight formulas give the weights that the user uses during performance of the exercises in the initial fitness assessment. In the example of Table 1, some of the exercises (e.g., Push Up and Sit Up) do not use weights. The weight formulas are expressed in terms of percentages of the body weight in Table 1. In other examples, the weight formulas could be expressed using other types of linear or nonlinear functions of the body weight.

TABLE 1

| | Resistance Training | |
|---|---|---|
| Movement Pattern | Exercise | Weight Formulas |
| Push | Push Up | 0 (Body weight) |
| Pull | Bent Row | 40% of body weight |
| Core | Sit Up | 0 (Body weight) |
| Squat | Front Squat | 50% of body weight |
| Hinge | Reverse Lunge | 40% of body weight |

During the initial fitness assessment, a user performs the exercises corresponding to the set of base movement patterns. The number of repetitions (or reps) that the user is able to complete for each exercise at the recommended weight is measured. Based on the number of exercise repetitions the user can complete, a rating can be assigned to each exercise for the user. Table 2 shows an example of ratings that can be applied to the exercises shown in Table 1. The ratings are based on fitness data from The American College of Sports Medicine (ACSM).

TABLE 2

| Movement | Rating | | | | |
|---|---|---|---|---|---|
| Pattern | Excellent | Good | Average | Poor | Very Poor |
| Horizontal | 48+ | 35-47 | 22-34 | 9-21 | 8 or fewer |
| Push | | | | | |
| Horizontal | 20+ | 15-19 | 10-14 | 5-9 | 0-4 |
| Pull | | | | | |
| Core | 48+ | 36-47 | 24-35 | 12-23 | 0-11 |
| Squat | 25 | 19-24 | 13-18 | 7-12 | 0-6 |
| Hinge | 24+ | 18-23 | 12-17 | 6-11 | 0-5 |

Using the results of the initial fitness assessment (e.g., the ratings for the exercises based on the user performance data), weights can be prescribed to the set of exercises in the user profile (see Example 2). Table 3 below shows an example of weight calculations that can be made for bent row or any other exercises having horizontal pull movement pattern as a primary movement pattern using the results of the initial fitness assessment for horizontal pull movement pattern. If an exercise has a horizontal pull movement pattern and the user performance rating for horizontal pull movement pattern is Excellent, for example, the weight calculation corresponding to Excellent will be used to determine the weight to prescribe to the exercise. In the weight calculations in Table 3, REPS is the number of repetitions determined for the horizontal pull movement pattern from the initial fitness assessment and BW is the body weight used during the initial fitness assessment.

TABLE 3

| Rating | Weight Calculation |
|---|---|
| Excellent | $( ( - \frac{\text{.1}}{3} ) \times R E P S + 1 ) \times B W$ |
| Good | $( ( - \frac{\text{.1}}{3} ) \times R E P S + . 95 ) \times B W$ |
| Average | $( ( - \frac{\text{.1}}{3} ) \times R E P S + . 85 ) \times B W$ |
| Poor | $( ( - \frac{\text{.1}}{3} ) \times R E P S + . 75 ) \times B W$ |
| Very Poor | $( ( - \frac{\text{.1}}{3} ) \times R E P S + . 65 ) \times B W$ |

### Example 9—Example Method Implementing Initial Weight Prescription for Resistance Training

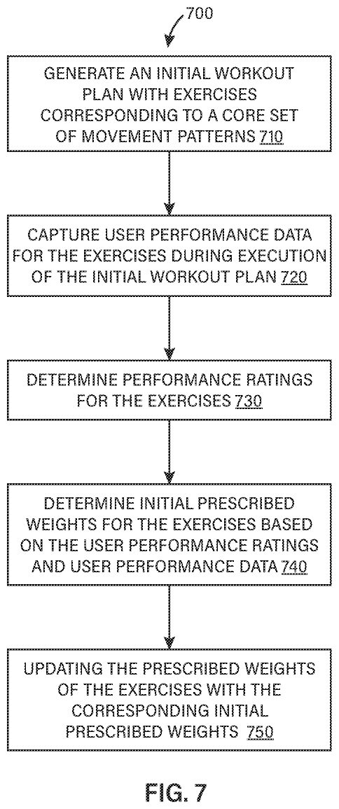

FIG. 7 is a flow diagram of an example method 700 of adaptive weight prescription for resistance training and can be performed, for example, by the system of FIG. 1. The method 700 can be performed prior to the method 200 in FIG. 2. The method 700 assumes that a user profile has been created for a user and that a set of exercises has been added to the user profile. The method 700 generates initial prescribed weights for the set of exercises.

At 710, the method can include generating an initial workout plan with exercises corresponding to a set of base movement patterns (see Example 8). For example, the initial workout plan can include the exercises shown in Table 1 in Example 8. The recommended weights for the exercises can be determined according to the weight formulas shown in Table 1 in Example 8. The weight formulas shown in Table 1 depend on the body weight of the user. The method can include requesting the body weight of the user or extracting the body weight of the user from the user profile.

At 720, the method can include capturing user performance data for the exercises during execution of the initial workout plan in an exercise environment by the user (see operation 220 in Example 3). The user performance data captured for each exercise can include the number of repetitions the user can complete for the exercise at the weight recommended for the exercise.

At 730, the method can include determining performance ratings for the exercises based on the user performance data (see Example 8). The performance ratings can be determined from lookup tables based on field data.

At 740, the method can include determining initial prescribed weights for a set of exercises associated with the user profile based on the performance ratings and the user performance data. For example, the method can have a set of rules to compute prescribed weight for an exercise E in the set of exercises. Each rule can be configured to generate a prescribed weight for a particular performance rating. Each rule can be a function of the body weight of the user and the number of repetitions of the exercise the user can complete (as determined from the user performance data). An example of a set of rules to compute prescribed weights is shown in Table 3 in Example 8.

At 750, the method includes updating the values of the prescribed weights of the exercises stored in the user profile with the corresponding initial prescribed weights determined in operation 740.

### Example 10—Exercise Coding

A set of exercises can be defined and stored in the exercises repository (see Example 2). Each exercise can have a set of coding attributes. In one example, the set of coding attributes can include exercise code, exercise type, primary movement pattern, secondary movement pattern, additional function, and exercise multiplier. An exercise can be uniquely identified by the exercise code. Table 4 shows examples of exercises with coding attributes. Each entry in Table 4 corresponds to a unique exercise.

TABLE 4

| | | | | Additional | |
|---|---|---|---|---|---|
| | | | | Function | |
| | | | Secondary | (Total Body | |
| | Exercise | | Movement | (T), | |
| | Type | Primary | Pattern | Compound | |
| Exercise | (Strength or | Movement | (Complex | (C), Accessory | Exercise |
| Code | Cardio) | Pattern | Exercise) | (A), None) | Multiplier |
| S(1) | Strength | Squat | None | None | 1 |
| HB(1) | Strength | Hinge: | None | None | 1 |
| | | Bilateral | | | |
| PC(1) | Strength | Push: Chest | None | None | 1 |
| PTA(.6) | Strength | Push: Tricep | None | Accessory | 0.6 |
| PUBA(.6) | Strength | Pull: Bicep | None | Accessory | 0.6 |
| ZC(0) | Cardio | Core | None | None | 0 |
| PUBPSX(0.6) | Strength | Pull: Bicep | Push | Compound | 0.6 |

As shown in Table 4, the exercise code can have a character portion and a number portion. The character portion is generated based on the values of the exercise type, primary movement pattern, secondary movement pattern, and additional function. The number portion is generated based on the exercise multiplier. For example, the first exercise entry in Table 4 has an exercise code S(1). The character portion of the exercise code is “S”, and the number portion of the exercise code is “1”, where the exercise multiplier is 1. “S” corresponds to Squat.

In some examples, the exercise type can be selected from a set S1={strength, cardio (Z)}. In some examples, the primary movement pattern can be selected from a set S2={Push: Chest (PC), Push: Shoulder (PS), Push: Tricep (PT), Push: Fly (PF), Pull: Back (PUL); Pull: Shoulder (PUS), Pull: Bicep (PUB), Core (C), Squat (S), Hinge: Unilateral (HU), Hinge: Bilateral (HB)}. In some examples, the secondary movement pattern can be selected from a set S3={None, Push: Chest (PC), Push: Shoulder (PS), Push: Tricep (PT), Push: Fly (PF), Pull: Back (PUL); Pull: Shoulder (PUS), Pull: Bicep (PUB), Core (C), Squat (S), Hinge: Unilateral (HU), Hinge: Bilateral (HB)}. In some examples, additional function can be selected from a set S4={None, Total Body (T), Compound (X), Accessory (A)}. The exercise multiplier for base movements is 1.0. Exercises using weight (other than body weight) have an exercise multiplier greater than 0 (e.g., equal to or greater than 0.1). Exercises using only body weight have an exercise multiplier of 0. The exercise multiplier adjusts the prescribed weight based on the biomechanics of the exercise.

The exercise coding creates a system that establishes equivalency between exercises. The character portion of the exercise code represent a particular combination of the exercise type, primary movement pattern, secondary movement, and additional function. Two unique exercises can have the same character portions but different number portions, different character portions but the same number portions, or different character portions and different number portions. Any two unique exercises with exercise codes having the same character portions can be swapped for each other in workouts. The exercise multiplier accounts for differences in the biomechanics of the exercises and allows this swapping. One usefulness of being able to swap exercises is to allow for flexibility in designing workouts.

When an exercise X is swapped for an exercise Y, the user performance data contained in exercise X can be leveraged in exercise Y for the user. For example, the value of the capability index for exercise Y can be set to the value of the capability index for exercise X when the swap is made. From the capability level for Y and using the exercise multiplier for Y, the prescribed weight for exercise Y can be determined. For example, the prescribed weight for exercise Y can be the capability index for exercise Y multiplied by the exercise multiplier for exercise Y.

Any exercise with a common code (e.g., and HB(x)) can be swapped with any other exercise having the code (e.g., HB(x)), where x represents the exercise multiplier. The multiplier does not need to be identical; it can be used to adjust the weight recommendation based on the previously demonstrated user capability for the specific movement pattern. For example, if a user is prescribed 40 pounds for HB(1), and an HB(0.5) exercise is swapped in, the weight recommended would be 20 pounds for the new exercise at the same number of repetitions.

The capability index can be used to initially categorize performance, which can be associated with a rule to calculate a weight prescription. As the user continues to do other exercises, the weight prescription can be related back to the capability index, which is associated with the rule that creates the weight recommendation and is modified by the exercise multiplier. The capability index can be ultimately associated with the fundamental movement patterns rather than specific exercises.

### Example 11—Exercise Structure

An exercise can have coding attributes (see Example 10) and performance attributes. Values for the coding attributes can be fixed. Values for the performance attributes can be adapted to user performance. The performance attributes can include, for example, prescribed weight, prescribed number of exercise repetitions, equipment type, prescribed workout volume, and capability index.

The prescribed weight is the recommended weight for performing the exercise.

The prescribed number of exercise repetitions is the recommended number of repetitions of the exercise in a workout. The number of repetitions can be divided into sets.

The equipment type is the type of resistance weight used in the exercise (e.g., the type of free weight, type of resistance machine, etc.). To standardize prescription of prescribed weights, a set of weight graduation levels can be defined for the equipment type. For illustration purposes, a single kettlebell can have a set of weight graduation levels WG={8 kg. 12 kg. 20 kg. 25 kg. 35 kg, 40 kg}. The prescribed weight can have a value selected from the set of weight graduation levels.

The workout volume is a measure of the total work involved in performing the exercise during a workout. For example, the prescribed workout volume can be defined as the prescribed weight multiplied by the prescribed number of exercise repetitions. The prescribed workout volume depends on the prescribed weight, which can change based on user performance of the exercise.

The capability index is a measure of a level of performance of the exercise, and it can be tracked per fundamental movement pattern (e.g., the fundamental movement patterns have respective capabilities indexes). Although an example uses a number 1 through 5 for the various movements, it could go up to any upper limit. The capability index can be based on performance at a prescribed weight and can change based on user performance of the exercise. In practice, the capability index can take the form of a general indication of initial capability (e.g., an integer in the range of 1-5 or the like); finer granularity can be supported.

When an exercise is newly added to a user profile, the prescribed weight attribute of the exercise can be null or have a default value. In some examples, the method 700 in FIG. 7 can be performed to assign an initial value to the prescribed weight attribute of the exercise that is based on an initial fitness assessment of the user.

### Example 12—Initial User Positioning

In any of the examples herein, initial user positioning (IUP) can be monitored and integrated into the technologies to facilitate subsequent form tracking and automatically counting repetitions. For example, a camera can generate images that are analyzed to determine whether the user is in a correct initial position. Such an arrangement can be helpful in aiding the user with the process of assuming a correct initial position, which can be an obstacle for new users. Subsequent activity such as form tracking and automatically counting repetitions via the camera can then be more reliably performed. However, the technologies can continue to function even if initial user positioning is not successful. Although correct positioning is a valuable feature and allows the user to take better advantage of the technologies, it is not essential.

Thus, in any of the examples herein, the technologies can comprise capturing an image of a user assuming an initial user position and confirming that the initial user position is correct. Such confirming can comprise comparing a determined position of the user based on the image against a correct position internally represented within software. In practice, a plurality of images, video images, or the like can be captured and analyzed.

Various user interfaces can be displayed when successful or when there are issues. The tracking technology may have difficulty if the user is not in the proper position, and some initial positioning assistance can overcome the difficulties. A user may decide that the tracking technology is not desired or wish to move on, so automatic repetition counting need not be used. Thus, manual counting can be used, and the user can still benefit from the other aspects of the technologies as described herein even if initial user positioning is not successful.

In an example embodiment, the application can evaluate plural different positions. First, vertical positioning is evaluated. A text prompt can be presented asking the user to stand facing the camera (e.g., with arms raised). Audio instructions can be provided (e.g., “Stand tall, facing the camera, with your arms extended up towards the ceiling.”). An earcon indicating that the system is looking at the user can be played. A user can opt out of form tracking if desired, resulting in a reminder to count repetitions manually.

If the user is determined to be in correct position based on the images, confirmation can be displayed and/or announced (e.g., “Looks good!”).

Horizontal positioning can follow (e.g., “Lie facedown, sideways to the camera. Extend your arms and straight out in front of you, with your legs extended straight out.”). Again, the earcon indicating that the system is looking at the user can be played.

If the user is determined to be in correct position based on the images, confirmation can be displayed and/or announced (e.g., “Looks good! Let's jump into the workout.”).

If the user is determined not to be in correct vertical or horizontal position, the process can pause to allow time for the user to get into position. If still not in correct position, a warning can be played (e.g., “Make sure you are visible in the frame”) and/or instructions can be repeated. If repeated failures are observed, the application can indicate that there are repeated issues. The user can move try again a threshold number of times, or manual counting of repetitions can be invoked.

### Example 13—Adaptive Strength Onboarding

In any of the examples herein, there a variety of approaches can be used to onboard new users into an adaptive program. For example, dummy or actual weights can be used to evaluate form. Alternatively, a series of questions can be asked to collect information from the user. In any event, steps can be taken to evaluate whether the user has proper form and what weight is appropriate.

In any of the examples herein, the technologies can comprise setting default weights values, receiving user responses to a series of questions, responsive to the user responses, adjusting the default weights values, and implementing the default weights values in the workout plan.

In an example embodiment, an onboarding process is triggered for the first adaptive program in weights modality. Default weights values can be put in place initially. Then, the user can be presented with a set of questions, and responsive to the user's answers to the questions, the initial weights values can be adjusted. The application can predict what the user is capable of doing. A user can skip questions if desired.

The weights values can then be used going into a pre-workout flow and ultimately when doing the workout (e.g., exercise).

### Example 14—Intelligent Form Correction

In any of the examples herein, intelligent form correction can be implemented. For example, a form correction prompt can be skipped or stopped if there is not enough time in the interval left for it to finish playing and for the user to process an integrate the correction (e.g., the exercise is toward the end of the interval). Form correction notifications (e.g., audio announcements) related to feedback keys can be spoken as many times as the feedback keys are received as long as they have been received at least one time before and if a notification for another feedback key is not currently being spoken. Form correction notifications can vary so that the same ones do not consistently crowd out others when multiply repeated. The form correction subsystem can send feedback keys with priority levels (e.g., injury prevention can be in the highest priority bucket). Random selection of remaining keys can be implemented.

In any of the examples herein, the technologies can comprise receiving an indication of a plurality of problems with form based on video captured during execution of the workout plan and selectively announcing a problem out of the problems with form based on remaining time in an interval, whether the problem has been observed before, or a priority of the problem. Such problems can be detected by comparing a determined position or motion of the user based on the video images against a correct position or motion internally represented within software.

In an embodiment, form correction can be received from a form correction subsystem that detects when form has deviated from a standard form by evaluating video images captured by a camera. However, if the form correction is received at the end of an interval (e.g., n seconds or less remain), form correction can be skipped. When form problems are detected, the form correction subsystem can output plural identified problems with form as feedback keys. Feedback keys that have been received before can be processed differently to prevent feedback fatigue. For example, if a text to speech prompt is currently being spoken, a notification for a feedback key that has been received before can be skipped to prevent cross talk. High priority keys can be selected, and keys can be randomly selected.

An carcon can be played to indicate that the system is about to speak, after which form correction related to the feedback key can be announced via audio (e.g., “If you can, try squatting a little deeper. Focus on engaging your glutes.”).

### Example—Computing Systems

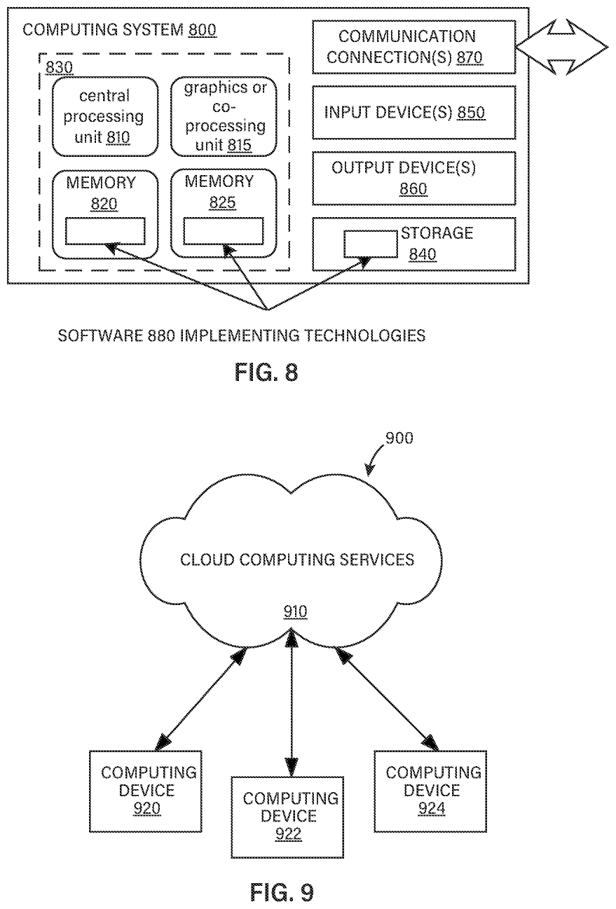

FIG. 8 depicts an example of a suitable computing system 800 in which the described innovations can be implemented. The computing system 800 is not intended to suggest any limitation as to scope of use or functionality of the present disclosure, as the innovations can be implemented in diverse computing systems.

With reference to FIG. 8, the computing system 800 includes one or more processing units 810, 815 and memory 820, 825. In FIG. 8, this basic configuration 830 is included within a dashed line. The processing units 810, 815 execute computer-executable instructions, such as for implementing the features described in the examples herein. A processing unit can be a general-purpose central processing unit (CPU), processor in an application-specific integrated circuit (ASIC), graphics processing unit (GPU), tensor processing unit (TPU), quantum processor, or any other type of processor. In a multi-processing system, multiple processing units execute computer-executable instructions to increase processing power. For example, FIG. 8 shows a central processing unit 810 as well as a graphics processing unit or co-processing unit 815. The tangible memory 820, 825 can be volatile memory (e.g., registers, cache, RAM), non-volatile memory (e.g., ROM, EEPROM, flash memory, etc.), or some combination of the two, accessible by the processing unit(s) 810, 815. The memory 820, 825 stores software 880 implementing one or more innovations described herein, in the form of computer-executable instructions suitable for execution by the processing unit(s) 810, 815.

A computing system 800 can have additional features. For example, the computing system 800 includes storage 840, one or more input devices 850, one or more output devices 860, and one or more communication connections 870, including input devices, output devices, and communication connections for interacting with a user. An interconnection mechanism (not shown) such as a bus, controller, or network interconnects the components of the computing system 800. Typically, operating system software (not shown) provides an operating environment for other software executing in the computing system 800, and coordinates activities of the components of the computing system 800.

The tangible storage 840 can be removable or non-removable, and includes magnetic disks, magnetic tapes or cassettes, CD-ROMs, DVDs, or any other medium which can be used to store information in a non-transitory way and which can be accessed within the computing system 800. The storage 840 stores instructions for the software 880 implementing one or more innovations described herein.

The input device(s) 850 can be an input device such as a keyboard, mouse, pen, or trackball, a voice input device, a scanning device, touch device (e.g., touchpad, display, or the like) or another device that provides input to the computing system 800. The output device(s) 860 can be a display, printer, speaker, CD-writer, or another device that provides output from the computing system 800, e.g., actuators or some mechanical devices like motors, 3D printers, and the like.

The communication connection(s) 870 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the context of computer-executable instructions, such as those included in program modules, being executed in a computing system on a target real or virtual processor (e.g., which is ultimately executed on one or more hardware processors). Generally, program modules or components include routines, programs, libraries, objects, classes, components, data structures, etc. that perform particular tasks or implement particular abstract data types. The functionality of the program modules can be combined or split between program modules as desired in various embodiments. Computer-executable instructions for program modules can be executed within a local or distributed computing system.

For the sake of presentation, the detailed description uses terms like “determine” and “use” to describe computer operations in a computing system. These terms are high-level descriptions for operations performed by a computer and should not be confused with acts performed by a human being. The actual computer operations corresponding to these terms vary depending on implementation.

### Example—Computer-Readable Media

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

Any of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing system to perform the method. The technologies described herein can be implemented in a variety of programming languages.

### Example—Cloud Computing Environment

FIG. 9 depicts an example cloud computing environment 900 in which the described technologies can be implemented, including, e.g., the systems described herein. The cloud computing environment 900 comprises cloud computing services 910. The cloud computing services 910 can comprise various types of cloud computing resources, such as computer servers, data storage repositories, networking resources, etc. The cloud computing services 910 can be centrally located (e.g., provided by a data center of a business or organization) or distributed (e.g., provided by various computing resources located at different locations, such as different data centers and/or located in different cities or countries).

The cloud computing services 910 are utilized by various types of computing devices (e.g., client computing devices), such as computing devices 920, 922, and 924. For example, the computing devices (e.g., 920, 922, and 924) can be computers (e.g., desktop or laptop computers), mobile devices (e.g., tablet computers or smart phones), or other types of computing devices. For example, the computing devices (e.g., 920, 922, and 924) can utilize the cloud computing services 910 to perform computing operations (e.g., data processing, data storage, and the like).

In practice, cloud-based, on-premises-based, or hybrid scenarios can be supported.

### Additional Examples

Additional examples based on principles described herein are enumerated below. Further examples falling within the scope of the subject matter can be configured by, for example, taking one feature of an example in isolation, taking more than one feature of an example in combination, or combining one or more features of one example with one or more features of one or more other examples. Any of the following can be implemented.

Clause 1. A computer-implemented method comprising:
generating a workout plan for a user having a user profile, the workout plan comprising an exercise selected from a set of exercises stored in the user profile, the exercise having a prescribed weight and a prescribed number of exercise repetitions;
capturing user performance data for the exercise during execution of the workout plan by the user in an exercise environment, the user performance data comprising an actual weight used in performance of the exercise by the user and an actual number of exercise repetitions performed by the user;
determining a new value for the prescribed weight based on a stored value of the prescribed weight in the user profile, the prescribed number of exercise repetitions, and the user performance data; and
adjusting the stored value of the prescribed weight in the user profile to the new value for the prescribed weight.

Clause 2. The computer-implemented method of Clause 1, wherein the exercise has an equipment type, wherein a set of weight graduation levels is defined for the equipment type, and wherein determining the new value comprises:
determining a weight graduation level in the set of weight graduation levels corresponding to the stored value; and
determining the new value relative to the weight graduation level corresponding to the stored value.

Clause 3. The computer-implemented method of any one of the Clauses 1-2, further comprising presenting the prescribed weight and the prescribed number of exercise repetitions in the exercise environment prior to capturing the user performance data for the exercise.

Clause 4. The computer-implemented method of any one of the Clauses 1-3, wherein capturing the user performance data for the exercise comprises receiving voice commands from the exercise environment and processing the voice commands to obtain at least a portion of the user performance data.

Clause 5. The computer-implemented method of any one of the Clauses 1-4, wherein capturing the user performance data from the exercise environment comprises capturing video images of the exercise environment and processing the video images to obtain at least a portion of the user performance data.

Clause 6. The computer-implemented method of any one of the Clauses 1-5, further comprising playing a video associated with the exercise in the exercise environment contemporaneously with capturing the user performance data for the exercise in the exercise environment.

Clause 7. The computer-implemented method of any one of the Clauses 1-6, wherein determining the new value for the prescribed weight comprises:
determining that the actual number of exercise repetitions is equal to or greater than the prescribed number of exercise repetitions;
determining that the actual weight used is equal to or greater than the prescribed weight;
determining that a ratio of an actual workout volume based on the actual weight and the actual number of exercise repetitions to a prescribed workout volume based on the prescribed weight and the prescribed number of exercise repetitions is equal to or greater than a threshold in a range from 1 to 1.5; and
selecting a weight graduation level from the set of weight graduation levels that is greater than the stored value of the prescribed weight as the new value for the prescribed weight.

Clause 8. The computer-implemented method of any one of the Clauses 1-7, wherein determining the new value for the prescribed weight comprises:
determining that the actual number of exercise repetitions is less than the prescribed number of exercise repetitions;
determining that the actual weight is the same as the prescribed weight;
determining that a ratio of an actual workout volume based on the actual weight and the actual number of exercise repetitions to a prescribed workout volume based on the prescribed weight and the prescribed number of exercise repetitions is equal to or less than a threshold in a range from 0.6 to 0.8; and
selecting a weight graduation level from the set of weight graduation levels that is lower than the stored value of the prescribed weight as the new value for the prescribed weight.

Clause 9. The computer-implemented method of any one of the Clauses 1-8, wherein determining the new value for the prescribed weight comprises:
determining that the actual number of exercise repetitions is equal to or greater than the prescribed number of exercise repetitions;
determining that the actual weight is lower than the prescribed weight; and
setting the new value for the prescribed weight to the actual weight.

Clause 10. The computer-implemented method of any one of the Clauses 1-9, wherein determining the new value for the prescribed weight comprises:
determining that the actual number of exercise repetitions is less than the prescribed number of exercise repetitions;
determining that the actual weight is lower than the prescribed weight;
determining that a ratio of an actual workout volume based on the actual weight and the actual number of exercise repetitions to a prescribed workout volume based on the prescribed weight and the prescribed number of exercise repetitions is greater than a threshold in a range from 0.4 to 0.6; and setting the new value for the prescribed weight to the actual weight.

Clause 11. The computer-implemented method of any one of the Clauses 1-10, wherein determining the new value for the prescribed weight comprises:
determining that the actual number of exercise repetitions is less than the prescribed number of exercise repetitions;
determining that the actual weight is lower than the prescribed weight;
determining that a ratio of an actual workout volume based on the actual weight and the actual number of exercise repetitions to a prescribed workout volume based on the prescribed weight and the prescribed number of exercise repetitions is less than or equal to a threshold in a range from 0.4 to 0.6; and
selecting a weight graduation level from the set of weight graduation levels that is lower than the actual weight as the new value for the prescribed weight.

Clause 12. The computer-implemented method of any one of the Clauses 1-11, wherein determining the new value for the prescribed weight comprises:
determining that the actual number of exercise repetitions is equal to or greater than the prescribed number of exercise repetitions;
determining that the actual weight is higher than the prescribed weight; and
setting the new value for the prescribed weight to the actual weight.

Clause 13. The computer-implemented method of any one of the Clauses 1-12, wherein determining the new value for the prescribed weight comprises:
determining that the actual number of exercise repetitions is less than the prescribed number of exercise repetitions;
determining that the actual weight is higher than the prescribed weight;
determining that a ratio of an actual workout volume based on the actual weight and the actual number of exercise repetitions to a prescribed workout volume based on the prescribed weight and the prescribed number of exercise repetitions is greater than a threshold in a range from 1 to 1.2; and
setting the new value for the prescribed weight to the actual weight.

Clause 14. The computer-implemented method of any one of the Clauses 1-13, wherein the exercise has an exercise code and an exercise multiplier associated with the exercise code, and further comprising:
determining a user capability index for the exercise based on the new value for the prescribed weight and the exercise multiplier; and
storing the user capability index in the user profile in association with the exercise.

Clause 15. The computer-implemented method of any one of the Clauses 1-14, further comprising:
generating an initial workout plan comprising a set of base movement patterns;
assigning prescribed weights to the set of base movement patterns based on a body weight of the user;
capturing initial user performance data for the set of base movement patterns during execution of the initial workout plan by the user, the initial user performance data comprising the number of exercise repetitions performed by the user for the set of base movement patterns; and
determining initial values for the prescribed weights of exercises in the set of exercises stored in the user profile based on the initial user performance data.

Clause 16. The computer-implemented method of claim 15, wherein determining the initial values for the prescribed weights of the exercises comprises:
determining a subset of the set of exercises having a primary movement pattern that matches a first movement pattern from the set of base movement patterns; and
determining initial values for the prescribed weights of the subset of the set of exercises based on a portion of the initial user performance data corresponding to the first movement pattern.

Clause 17. The computer-implemented method of any one of the Clauses 1-16, further comprising:
capturing an image of a user assuming an initial user position; and
confirming that the initial user position is correct based on the image.

Clause 18. The computer-implemented method of any one of the Clauses 1-17, further comprising:
setting default weights values;
receiving user responses to a series of questions;
responsive to the user responses, adjusting the default weights values; and
implementing the default weights values in the workout plan.

Clause 19. The computer-implemented method of any one of the Clauses 1-18, further comprising:
receiving an indication of a plurality of problems with form based on video captured during execution of the workout plan; and
selectively announcing a problem out of the problems with form based on remaining time in an interval, whether the problem has been observed before, or a priority of the problem.

Clause 20. A computing system comprising:
at least one hardware processor;
at least one memory coupled to the at least one hardware processor; and
one or more non-transitory computer-readable media having stored therein computer-executable instructions that, when executed by the computing system, cause the computing system to perform:
generating a workout plan for a user having a user profile, the workout plan comprising an exercise selected from a set of exercises stored in the user profile, the exercise having a prescribed weight and a prescribed number of exercise repetitions;
capturing user performance data for the exercise during execution of the workout plan by the user in an exercise environment, the user performance data comprising an actual weight used in performance of the exercise by the user and an actual number of exercise repetitions performed by the user;
determining a new value for the prescribed weight based on a stored value of the prescribed weight in the user profile, the prescribed number of exercise repetitions, and the user performance data; and
adjusting the stored value of the prescribed weight in the user profile to the new value for the prescribed weight.

Clause 21. One or more non-transitory computer-readable media storing computer-executable instructions that when executed cause a computing system to perform operations comprising:
generating a workout plan for a user having a user profile, the workout plan comprising an exercise selected from a set of exercises stored in the user profile, the exercise having a prescribed weight and a prescribed number of exercise repetitions;
capturing user performance data for the exercise during execution of the workout plan by the user in an exercise environment, the user performance data comprising an actual weight used in performance of the exercise by the user and an actual number of exercise repetitions performed by the user, wherein capturing the user performance data comprising capturing video images of the exercise environment during execution of the workout plan by the user in the exercise environment and processing the video images to obtain at least a portion of the user performance data;
determining a new value for the prescribed weight based on a stored value of the prescribed weight in the user profile, the prescribed number of exercise repetitions, and the user performance data; and
adjusting the stored value of the prescribed weight in the user profile to the new value for the prescribed weight.

Clause 22. The one or more non-transitory computer-readable medium of Clause 21, wherein the exercise has an exercise code and an exercise multiplier associated with the exercise code, and wherein the operations further comprise:
determining a user capability index for the exercise based on the new value for the prescribed weight and the exercise multiplier; and
storing the user capability index in the user profile in association with the exercise.

Clause 23. The one or more non-transitory computer-readable medium of Clause 21, wherein the operations further comprise:
generating an initial workout plan comprising a set of base movement patterns;
assigning prescribed weights to the set of base movement patterns based on a body weight of the user;
capturing initial user performance data for the set of base movement patterns during execution of the initial workout plan by the user, the initial user performance data comprising the number of exercise repetitions performed by the user for the set of base movement patterns; and
determining a subset of the set of exercises having a primary movement pattern that matches a first movement pattern from the set of base movement patterns; and
determining initial values for the prescribed weights of the subset of the set of exercises based on a portion of the initial user performance data corresponding to the first movement pattern.

### Example Implementation

Although the operations of some of the disclosed methods are described in a particular, sequential order for convenient presentation, such manner of description encompasses rearrangement, unless a particular ordering is required by specific language set forth herein. For example, operations described sequentially can in some cases be rearranged or performed concurrently.

### Example Alternatives

The technology has been described with a selection of implementations and examples, but these preferred implementations and examples are not to be taken as limiting the scope of the technology since many other implementations and examples are possible that fall within the scope of the disclosed technology. The scope of the disclosed technology includes what is covered by the scope and spirit of the following claims.

1. A computer-implemented method comprising:
generating a workout plan for a user having a user profile, the workout plan comprising an exercise selected from a set of exercises stored in the user profile, the exercise having a prescribed weight and a prescribed number of exercise repetitions;
capturing user performance data for the exercise during execution of the workout plan by the user in an exercise environment, the user performance data comprising an actual weight used in performance of the exercise by the user and an actual number of exercise repetitions performed by the user;
determining a new value for the prescribed weight based on a stored value of the prescribed weight in the user profile, the prescribed number of exercise repetitions, and the user performance data; and
adjusting the stored value of the prescribed weight in the user profile to the new value for the prescribed weight.

2. The computer-implemented method of claim 1, wherein the exercise has an equipment type, wherein a set of weight graduation levels is defined for the equipment type, and wherein determining the new value comprises:
determining a weight graduation level in the set of weight graduation levels corresponding to the stored value; and
determining the new value relative to the weight graduation level corresponding to the stored value.

3. The computer-implemented method of claim 1, further comprising presenting the prescribed weight and the prescribed number of exercise repetitions in the exercise environment prior to capturing the user performance data for the exercise.

4. The computer-implemented method of claim 1, wherein capturing the user performance data for the exercise comprises receiving voice commands from the exercise environment and processing the voice commands to obtain at least a portion of the user performance data.

5. The computer-implemented method of claim 1, wherein capturing the user performance data from the exercise environment comprises capturing video images of the exercise environment and processing the video images to obtain at least a portion of the user performance data.

6. The computer-implemented method of claim 1, further comprising playing a video associated with the exercise in the exercise environment contemporaneously with capturing the user performance data for the exercise in the exercise environment.

7. The computer-implemented method of claim 1, wherein determining the new value for the prescribed weight comprises:
determining that the actual number of exercise repetitions is equal to or greater than the prescribed number of exercise repetitions;
determining that the actual weight used is equal to or greater than the prescribed weight;
determining that a ratio of an actual workout volume based on the actual weight and the actual number of exercise repetitions to a prescribed workout volume based on the prescribed weight and the prescribed number of exercise repetitions is equal to or greater than a threshold in a range from 1 to 1.5; and
selecting a weight graduation level from the set of weight graduation levels that is greater than the stored value of the prescribed weight as the new value for the prescribed weight.

8. The computer-implemented method of claim 1, wherein determining the new value for the prescribed weight comprises:
determining that the actual number of exercise repetitions is less than the prescribed number of exercise repetitions;
determining that the actual weight is the same as the prescribed weight;
determining that a ratio of an actual workout volume based on the actual weight and the actual number of exercise repetitions to a prescribed workout volume based on the prescribed weight and the prescribed number of exercise repetitions is equal to or less than a threshold in a range from 0.6 to 0.8; and
selecting a weight graduation level from the set of weight graduation levels that is lower than the stored value of the prescribed weight as the new value for the prescribed weight.

9. The computer-implemented method of claim 1, wherein determining the new value for the prescribed weight comprises:
determining that the actual number of exercise repetitions is equal to or greater than the prescribed number of exercise repetitions;
determining that the actual weight is lower than the prescribed weight; and
setting the new value for the prescribed weight to the actual weight.

10. The computer-implemented method of claim 1, wherein determining the new value for the prescribed weight comprises:
determining that the actual number of exercise repetitions is less than the prescribed number of exercise repetitions;
determining that the actual weight is lower than the prescribed weight;
determining that a ratio of an actual workout volume based on the actual weight and the actual number of exercise repetitions to a prescribed workout volume based on the prescribed weight and the prescribed number of exercise repetitions is greater than a threshold in a range from 0.4 to 0.6; and
setting the new value for the prescribed weight to the actual weight.

11. The computer-implemented method of claim 1, wherein determining the new value for the prescribed weight comprises:
determining that the actual number of exercise repetitions is less than the prescribed number of exercise repetitions;
determining that the actual weight is lower than the prescribed weight;
determining that a ratio of an actual workout volume based on the actual weight and the actual number of exercise repetitions to a prescribed workout volume based on the prescribed weight and the prescribed number of exercise repetitions is less than or equal to a threshold in a range from 0.4 to 0.6; and
selecting a weight graduation level from the set of weight graduation levels that is lower than the actual weight as the new value for the prescribed weight.

12. The computer-implemented method of claim 1, wherein determining the new value for the prescribed weight comprises:
determining that the actual number of exercise repetitions is equal to or greater than the prescribed number of exercise repetitions;
determining that the actual weight is higher than the prescribed weight; and
setting the new value for the prescribed weight to the actual weight.

13. The computer-implemented method of claim 1, wherein determining the new value for the prescribed weight comprises:
determining that the actual number of exercise repetitions is less than the prescribed number of exercise repetitions;
determining that the actual weight is higher than the prescribed weight;
determining that a ratio of an actual workout volume based on the actual weight and the actual number of exercise repetitions to a prescribed workout volume based on the prescribed weight and the prescribed number of exercise repetitions is greater than a threshold in a range from 1 to 1.2; and
setting the new value for the prescribed weight to the actual weight.

14. The computer-implemented method of claim 1, wherein the exercise has an exercise code and an exercise multiplier associated with the exercise code, and further comprising:
determining a user capability index for the exercise based on the new value for the prescribed weight and the exercise multiplier; and
storing the user capability index in the user profile in association with the exercise.

15. The computer-implemented method of claim 1, further comprising:
generating an initial workout plan comprising a set of base movement patterns;
assigning prescribed weights to the set of base movement patterns based on a body weight of the user;
capturing initial user performance data for the set of base movement patterns during execution of the initial workout plan by the user, the initial user performance data comprising the number of exercise repetitions performed by the user for the set of base movement patterns; and
determining initial values for the prescribed weights of exercises in the set of exercises stored in the user profile based on the initial user performance data.

16. The computer-implemented method of claim 15, wherein determining the initial values for the prescribed weights of the exercises comprises:
determining a subset of the set of exercises having a primary movement pattern that matches a first movement pattern from the set of base movement patterns; and
determining initial values for the prescribed weights of the subset of the set of exercises based on a portion of the initial user performance data corresponding to the first movement pattern.

17. The computer-implemented method of claim 1, further comprising:
capturing an image of a user assuming an initial user position; and
confirming that the initial user position is correct based on the image.

18. The computer-implemented method of claim 1, further comprising:
setting default weights values;
receiving user responses to a series of questions;
responsive to the user responses, adjusting the default weights values; and
implementing the default weights values in the workout plan.

19. The computer-implemented method of claim 1, further comprising:
receiving an indication of a plurality of problems with form based on video captured during execution of the workout plan; and
selectively announcing a problem out of the problems with form based on remaining time in an interval, whether the problem has been observed before, or a priority of the problem.

20. A computing system comprising:
at least one hardware processor;
at least one memory coupled to the at least one hardware processor; and
one or more non-transitory computer-readable media having stored therein computer-executable instructions that, when executed by the computing system, cause the computing system to perform:
generating a workout plan for a user having a user profile, the workout plan comprising an exercise selected from a set of exercises stored in the user profile, the exercise having a prescribed weight and a prescribed number of exercise repetitions;
capturing user performance data for the exercise during execution of the workout plan by the user in an exercise environment, the user performance data comprising an actual weight used in performance of the exercise by the user and an actual number of exercise repetitions performed by the user;
determining a new value for the prescribed weight based on a stored value of the prescribed weight in the user profile, the prescribed number of exercise repetitions, and the user performance data; and
adjusting the stored value of the prescribed weight in the user profile to the new value for the prescribed weight.

21. One or more non-transitory computer-readable media storing computer-executable instructions that when executed cause a computing system to perform operations comprising:
generating a workout plan for a user having a user profile, the workout plan comprising an exercise selected from a set of exercises stored in the user profile, the exercise having a prescribed weight and a prescribed number of exercise repetitions;
capturing user performance data for the exercise during execution of the workout plan by the user in an exercise environment, the user performance data comprising an actual weight used in performance of the exercise by the user and an actual number of exercise repetitions performed by the user, wherein capturing the user performance data comprising capturing video images of the exercise environment during execution of the workout plan by the user in the exercise environment and processing the video images to obtain at least a portion of the user performance data;
determining a new value for the prescribed weight based on a stored value of the prescribed weight in the user profile, the prescribed number of exercise repetitions, and the user performance data; and
adjusting the stored value of the prescribed weight in the user profile to the new value for the prescribed weight.

22. The one or more non-transitory computer-readable media of claim 21, wherein the exercise has an exercise code and an exercise multiplier associated with the exercise code, and wherein the operations further comprise:
determining a user capability index for the exercise based on the new value for the prescribed weight and the exercise multiplier; and
storing the user capability index in the user profile in association with the exercise.

23. The one or more non-transitory computer-readable media of claim 21, wherein the operations further comprise:
generating an initial workout plan comprising a set of base movement patterns;
assigning prescribed weights to the set of base movement patterns based on a body weight of the user;
capturing initial user performance data for the set of base movement patterns during execution of the initial workout plan by the user, the initial user performance data comprising the number of exercise repetitions performed by the user for the set of base movement patterns; and
determining a subset of the set of exercises having a primary movement pattern that matches a first movement pattern from the set of base movement patterns; and
determining initial values for the prescribed weights of the subset of the set of exercises based on a portion of the initial user performance data corresponding to the first movement pattern.
